(19) United States
(12) Patent Application Publication
(10) Pub. No.: US 2004/0254100 A1
(43) Pub. Date: Dec. 16, 2004

(54) Use of thiostrepton as an anti-mycobacterial agent

(76) Inventors: Mary W. Vermeulen, Ipswich, MA (US); Jiayi Wu, Brookline, MA (US)

Correspondence Address: CLARK & ELBING LLP, 101 FEDERAL STREET, BOSTON, MA, 02110, US

(21) Appl. No.: 10/182,658

(22) PCT Filed: Jan. 31, 2001
(86) PCT No.: PCT/US01/03136

Related U.S. Application Data
(60) Provisional application No. 60/179,203, filed on Jan. 31, 2000.

(51) Int. Cl. A61K 38/12

(52) U.S. Cl. 514/10

(57) ABSTRACT

Methods of using thiostrepton to treat or prevent mycobacterial infection are provided.

This invention relates to methods of treating and preventing mycobacterial infection.

### BACKGROUND OF THE INVENTION

Thiostrepton is a naturally-occurring antibacterial agent isolated from the soil microorganism Streptomyces aureus. Thiostrepton was first described in the scientific literature in 1955 (Pagano et al., Antibiot. Ann. 1955-56; 554), was patented in Britain in 1958 (British Patent No. 795,570), and was patented in the United States by Olin Mathieson in 1961 (U.S. Pat. Nos. 2,982,689 and 2,982,698). Preparation of thiostrepton was also patented by Olin Mathieson in 1965 (U.S. Pat. No. 3,181,995). The mechanism of action of thiostrepton, disruption of protein synthesis, was described in 1971 (Cannon et al., FEBS Letters 18; 1). The Merck Index reports that thiostrepton has been used as a veterinary antibacterial agent. The structure of thiostrepton is shown in FIG. 1.

### SUMMARY OF THE INVENTION

We have shown that thiostrepton is a potent inhibitor of growth of the mycobacterium Mycobactertum tuberculosis.

Accordingly, the invention provides a method of treating or preventing mycobacterial infection (e.g., tuberculosis) in a patient by administering thiostrepton to the patient. The thiostrepton can be administered at a dosage of, for example, 75-750, 150-500, or 250-400 mg/day, and can be administered parenterally (e.g., intravenously, intrapulmonarily, intraperitoneally, or subcutaneously) or mucosally (e.g., orally). The invention also includes the use of thiostrepton in the preparation of a medicament for treating or preventing mycobacterial infection (e.g., tuberculosis), as discussed above.

Other features and advantages of the invention will be apparent from the following detailed description, the drawings, and the claims.

### BRIEF DESCRIPTION OF THE DRAWINGS

FIG. 1 is a schematic representation of the chemical structure of thiostrepton.

### DETAILED DESCRIPTION

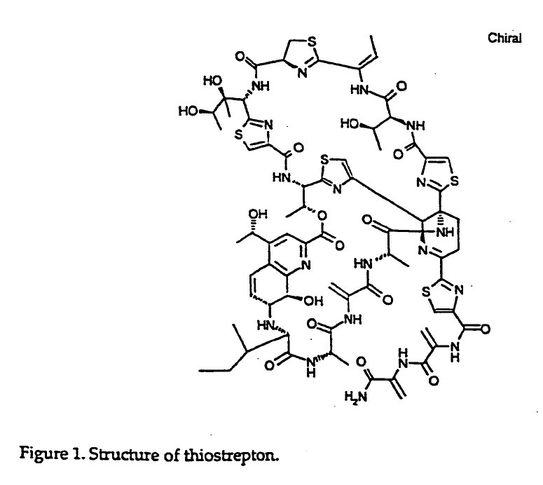

The invention provides methods of treating mycobacterial infection, e.g., tuberculosis, in a patient by administering thiostrepton to the patient. The compound can be delivered to a patient by any of several standard routes, for example, by mucosal (e.g., oral) or parenteral (e.g., intravenous, intrapulmonary, intraperitoneal, or subcutaneous) routes. The compound can be administered at a dosage and in a regimen that are determined to be appropriate by one of skill in the art based on, for example, the severity of infection or whether the treatment is being carried out for prophylaxis (e.g., in a subject at risk of exposure to a mycobacterium, such as M. tuberculosis). The compound can thus be administered, for example, at a dosage of 75-750 mg/day, e.g., 150-500 mg/day or 250-400 mg/day, for up to 6 months (e.g., for 1 week to 3 months or 2 weeks to a month), until symptoms of the infection have decreased in severity, the infection is cleared, the patient is no longer at risk for relapse, or, in the case of prophylaxis, the subject is no longer at risk of exposure to a mycobacterium. The compound is typically administered in a pharmaceutically acceptable formulation, e.g., physiological saline or sterile water, each of which may include 5% glucose. Additional formulations that can be used are well known in the art (see, e.g., Remington's Pharmaceutical Sciences, (18^{th} edition), ed. A. Gennaro, 1990, Mack Publishing Company, Easton, Pa.). Thiostrepton can be purchased from any of a number of commercial vendors, such as, for example, Calbiochem, Fluka, ICN, or Sigma, and its structure is shown in FIG. 1.

Use of thiostrepton to treat mycobacterial infection according to the invention is based on experimental results obtained using the following methods.

Discovery of Novel Use of Thiostrepton

During screening for natural products that inhibit growth of broth cultures of Mycobacterium tuberculosis (strain Ra), we discovered that thiostrepton is a potent inhibitor of such growth, having an IC₉₉ of between 30 and 100 nM. We also observed that thiostrepton is effective in our Bactec-based assay of M. tuberculosis, which involves infecting the murine alveolar macrophage-like cell line MH-S (see below), having an IC₅₀ of 30 nM.

Because mycobacteria grow very slowly, we employ assays that are faster than conventional turbidometric (broth-based) or colony count (plate-based) microbiological methods. We have taken advantage of specialized instrumentation (Bactec 460; Becton-Dickinson) to speed determination of mycobacterial growth rates two- to three-fold. The principle is to culture mycobacteria in a specialized medium that contains ¹⁴C-palmitate as an energy source. As the organisms grow, they convert the palmitate into ¹⁴CO₂. A daily sampling of the air above the cultures facilitates quantification of ¹⁴CO₂ concentrations, and shows whether the mycobacteria are growing or dying. Multiple readings can be combined to generate growth curves.

Detection and Quantification of Mycobacterial Growth

Sterile Bactec media vials are first monitored on the Bactec 460, both to establish a CO₂-enriched atmosphere in the vial and to detect any contamination of the media. A vial showing an initial growth index (GI; range 1-999) of 20 or more is assumed to be contaminated and is discarded. Next, one vial is inoculated with 0.1 ml of H37Ra from a culture that has been growing in Middlebrook 7H9 mycobacterial medium, and is incubated at 37±1° C. Measurements are obtained daily until the GI is ≧999. This vial then serves as the source of standard inoculum for an experiment. Prior to use, a small aliquot of inoculum is examined microscopically with Gram stain to assure the absence of contamination. With a tuberculin syringe, Bactec media vials are each inoculated with 0.1 ml of the bacterial suspension, resulting in a final concentration of between 10⁴ and 10⁵ colony-forming units (CFU) per ml. Vials are then incubated at 37±1° C. Measurements are obtained daily on the Bactec, and are graphed as a function of time to show growth rates.

Effect of Compounds on Mycobacterial Growth

The assay described above is modified slightly to assess the influence of compounds on mycobacterial growth. Just prior to inoculation of multiple vials with organisms, test compounds are added to the vials. Replicate control vials receive no drugs, and only 1% as many organisms (i.e., about 10² to 10³ CFU/ml, prepared as a 1:100 dilution of the stock inoculum into sterile Bactec diluting fluid). As is described above, vials are incubated at 37±1° C., and measurements are obtained at 24±2 hour intervals on the Bactec, until the 1% control vial has a GI reading of ≧30. Thiostrepton was identified as having anti-mycobacterial activity by use of this method.

Intracellular Assay

Disease-causing mycobacteria typically live inside of macrophages, a cell type that normally functions to recognize, ingest, and destroy foreign substances, such as bacteria. Thus, for patients with tuberculosis, a successful drug must inhibit organisms residing inside this host cell, which is a more challenging situation than exists for most antibiotics. We have developed an assay that allows us to screen for substances that inhibit growth of mycobacteria inside macrophages. This assay is described further, as follows.

Macrophages

The murine alveolar (lung) macrophage cell line MH-S was obtained from the American Type Culture Collection. These adherent cells are maintained in standard RPMI-1640 medium, supplemented with 10% fetal bovine serum (FBS), and passaged after detachment with EDTA-trypsin. For the assay, 5×10⁵ cells/well are seeded in a 96-well plate, suspended in RPMI-1640 with 10% FBS, and allowed to adhere overnight at 37° C.

Mycobacteria

From a log-phase culture of M. tuberculosis H37Ra, mycobacteria are added to the plated MH-S cells at a ratio of 10 mycobacteria to one macrophage. Infection is allowed to proceed for 2 hours, after which unbound mycobacteria are removed with two washes in HBSS. Cultures are examined microscopically to ensure that the washes are thorough enough.

Compounds

Test compounds are then added to duplicate wells of infected cells, and the incubation is continued for 24 hours, after which the MH-S cells are lysed by addition of 0.05% saponin, which does not affect mycobacterial survival or subsequent extracellular growth. Duplicate Bactec bottles are each inoculated with 0.1 ml of this material, and are incubated at 37° C. for several days, with Bactec growth index readings obtained daily, by measuring ¹⁴CO₂ released into the air above mycobacterial cultures from metabolism of [¹⁴C]palmitate in the Bactec medium. Growth rates of these cultures are contrasted to those of control cultures that are treated identically, except that no inhibitory compounds are added to the controls. An IC₅₀ is determined by comparing the growth of a standard inoculum of M. tuberculosis in untreated MH-S cells to growth in cells exposed to test compound.

What is claimed is:

1. A method of treating or preventing mycobacterial infection in a patient, said method comprising administering thiostrepton to said patient.

2. The method of claim 1, wherein said mycobacterial infection is tuberculosis.

3. The method of claim 1, wherein 75-750, 150-500, or 250-400 mg/day of thiostrepton is administered to said patient.

4. The method of claim 1, wherein said thiostrepton is parenterally, intravenously, intrapulmonarily, mucosally, or orally administered to said patient.

5. Use of thiostrepton in the preparation of a medicament for treating or preventing mycobacterial infection in a patient.

6. The use of claim 5, wherein said mycobacterial infection is tuberculosis.

7. The use of claim 5, wherein said medicament is formulated for administration of 75-750, 150-500, or 250-400 mg/day of thiostrepton to said patient. 8. The use of claim 5, wherein said medicament is formulated for parenteral, intravenous, intrapulmonary, mucosal, or oral administration to said patient.
